(19) United States
(12) Patent Application Publication
(10) Pub. No.: US 2005/0152996 A1
(43) Pub. Date: Jul. 14, 2005

(54) Extracts of Mimulus aurantiacus for treating psoriasis and repelling insects

(76) Inventor: Donald Butler, Escondido, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/999,241

(22) Filed: Nov. 30, 2004

Related U.S. Application Data
(60) Provisional application No. 60/526,032, filed on Dec. 1, 2003.

(51) Int. Cl. A61K 35/78

(52) U.S. Cl. 424/725

(57) ABSTRACT

The present invention provides extracts of the plant Mimulus aurantiacus for use in the treatment of a side variety of skin ailments, and in particular to alleviating psoriasis. The present invention includes compositions that include Mimulus aurantiacus extracts that can be used topically, and can be formulated as lotions, oils, shampoos, soaps, sprays, creams, salves, foams, or gels. The present invention also provides extracts of the plant Mimulus aurantiacus for use as insect repellants that are biodegradable and non-toxic to humans and animals. The present invention includes insect-repelling compositions that include Mimulus aurantiacus extracts that can be used on plants that can be formulated as powders, sprays or mulches. The present invention also includes insect-repelling compositions that include Mimulus aurantiacus extracts that can be used on animals or humans and can be formulated as lotions, sprays, shampoos, soaps, creams, salves, foams, or gels.

This application claims benefit of priority to U.S. Provisional Application 60/526,032, entitled “Extracts of Mimulus Aurantiacus for Treating Psoriasis and Repelling Insects” filed Dec. 1, 2003 and herein incorporated by reference.

### BACKGROUND OF THE INVENTION

The present invention relates to the field of topical treatment of skin ailments, and in particular to the use of certain plant extracts in topically treating skin ailments such as psoriasis, and in preventing allergic contact dermatitis due to plant toxins such as urushiol. The present invention also relates to the field of insect repellants, and in particular to insect repellants that comprise plant extracts that are biodegradable and non-toxic to humans and animals.

Psoriasis is a chronic disease of the skin that causes raised red patches that itch, often painfully. In plaque psoriasis, the most common form, the red plaques are often covered with silvery scales of dead skin, and are often localized to the knees, elbows, and torso. Other types of psoriasis are erythrodermic psoriasis, that covers most of the body, guttate psoriasis, concentrated on the torso, palmoplantar psoriasis, localized to the palms or the hands and soles of the feet, and pustular psoriasis, in which pustules develop on the plaques. Psoriasis, which can be mild, moderate, or severe, is believed to be an immune system disorder in which the patients T cells attack dermal cells, leading to inflammation and hyperproliferation of the skin cells. The symptoms may subside or even go into remission and then can recur or increase in severity.

Current therapies seek to dampen the immune response, reduce skin cell proliferation, relieve itching, or a combination of these strategies. Therapies can be topical, systemic, or light-based. Many of the currently available therapies are either hazardous (UVA treatment of the skin), or of limited efficacy.

Poison ivy, poison oak, and poison sumac (as well as some other plant species) contain resin that contains urushiol, a toxin that comprises a mixture of catechol derivatives. These catechol derivatives comprise 1,2 dihydroxy-benzene having a 15 or 17 carbon atom aliphatic side chain. These molecules can penetrate the skin, become metabolized to reactive quinone derivatives, and react with skin proteins such as keratin forming complexes that stimulate the immune system. The allergic contact dermatitis that results causes an itchy rash that can result in painful swelling and blisters. Various treatments, from topical lotions to administration of corticosteroids can be used to alleviate the itching and swelling of the urushiol-induced rash, however, they may have limited effectiveness or undesirable side effects.

Mimulus aurantiacus, commonly called the “Sticky Monkeyflower” is an herbaceous plant native to the West Coast of North America, ranging from northern California to Baja. The leaves, stems, and flowers of the plants are reported to be edible; reportedly native Americans used the leaves as a salad. Native Americans also reportedly used the leaves as a poultice for rope burns on the palms of the hands.

The leaves of Mimulus aurantiacus have a sticky resin on the surface. The leaf surface resin has been found to contain several flavonoids. The predominant leaf surface flavonoids were characterized as the terpenoid flavanones mimulone, diplacone, 3′-O methyldiplacone, diplacol, and 3′-O methyldiplacol. Less abundant flavonoids of the leaf surface were determined to be 3-geranyl-4-hydroxy-6-(2-hydroxypropyl)-2-pyrone, and eriodictyol 7, 3′ dimethyl ether. The structures of these molecules are provided in: Hare (2002) ‘Geographic and geneic variation in the leaf surface resin components of Mimulus aurantiacus from southern California’ Biochemical Systematics and Ecology 30: 281-296.

Proposed roles for the leaf surface flavonoids of Mimulus aurantiacus include protection from dessication, UV light protection, and resistance to insects.

Damage or destruction of plants due to insects can be vexing and also costly. Many insecticides and insect repellants in current use are toxic to humans, animals, fish, or amphibians, and can pose a hazard to the user as well as to the environment. Insect repellants for human and animal use such as DEET may pose risks as well, are often unsuitable for use on children or by pregnant women, and can persist in the environment. There is a need for insect repellants for both plants and animals that are effective, non-toxic, and compatible with the natural environment.

### BRIEF SUMMARY OF THE INVENTION

The present invention recognizes that there is a need for a safe and effective treatment for psoriasis and other skin ailments that has minimal or nonexistent side effects. The present invention provides compositions and methods for treating skin ailments.

In one aspect, the present invention comprises a composition for the topical treatment of a skin ailment comprising an extract of the leaves, stems, roots, flowers, seeds, or any combination thereof of Mimulus aurantiacus. The extract can be made by rinsing, soaking, or heating the leaves, stems, roots, flowers, seeds, or any combination thereof of Mimulus aurantiacus in a liquid, such as a liquid that contains water, at least one oil, acetone, or at least one alcohol. The extract can also be made by heating the leaves, stems, roots, flowers, seeds, or any combination thereof of Mimulus aurantiacus in a liquid comprising water, at least one oil, acetone, or at least one alcohol. In addition to the extract of Mimulus aurantiacus, the composition can comprise any of various compounds or substances such as emollients, oils, waxes, paraffin, antioxidants, or additional plant extracts. The composition can be used to treat any skin ailment, including psoriasis, eczema, tinea, acne, insect bites, and dermatitis. The composition can be formulated as a lotion, oil, shampoo, soap, spray, cream, salve, foam, or gel.

Another aspect of the present invention is a method of treating a skin ailment, comprising administering a composition of the present invention that comprises an extract of Mimulus aurantiacus to the skin of a subject. The skin ailment can be an ailment such as psoriasis, eczema, tinea, acne, reaction to insect bites, dandruff, seborrheic dermatitis, perioral dermatitis, atopic, dermatitis, or contact dermatitis. The composition can be administered from three times a day to twice weekly.

The present invention also recognizes there is a need for effective insect and herbivore repellants that are non-toxic and nonirritating to humans and animals, and are environment-compatible. A third aspect of the present invention is a composition for repelling insects or herbivores comprising an extract of the leaves, stems, roots, flowers, seeds, or combination thereof, of Mimulus aurantiacus. The extract can be made by rinsing, soaking, or heating the leaves, stems, roots, flowers, seeds, or any combination thereof of Mimulus aurantiacus in a liquid, such as a liquid that contains water, at least one oil, acetone, or at least one alcohol. The extract can also be made by heating the leaves, stems, roots, flowers, seeds, or any combination thereof of Mimulus aurantiacus in a liquid comprising water, at least one oil, acetone, or at least one alcohol. In addition to the extract of Mimulus aurantiacus, the composition can comprise any of various compounds or substances such as emollients, oils, surfactants, moisturizers, antioxidants, or one or more additional plant extracts. The composition can be formulated as a lotion, spray, shampoo, soap, cream, salve, foam, or gel.

A fourth aspect of the present invention is a method of repelling or eradicating insects comprising applying a composition of the present invention to an animal or human subject. A fifth aspect of the present invention is a method of repelling insects or herbivores comprising applying a composition of the present invention to a plant.

A sixth aspect of the invention is compositions for increasing tolerance of an individual for a toxin, such as a plant toxin such as, but not limited to, poison ivy (Toxicodendron (or Rhus) rydbergii, Toxicodendron (or Rhus) radicans) poison oak (Toxicodendron (or Rhus) diversilobum, Toxicodendron (or Rhus) toxicarium) and poison sumac (Toxicodendron (or Rhus) vernix). The compositions preferably include: an extract of Mimulus aurantiacus and at least one plant toxin. The composition can further comprise any of various compounds or substances such as emollients, oils, surfactants, moisturizers, antioxidants, or one or more additional plant extracts. The composition can be formulated as a lotion, spray, cream, salve, foam, or gel.

A seventh aspect of the present invention is a method of increasing tolerance of an individual for a plant toxin. The method includes: applying a lotion, spray, cream, salve, foam, or gel that comprises an extract of Mimulus aurantiacus and a plant toxin to the skin of an individual that is sensitive to the plant toxin. Preferably, the method includes multiple applications of the composition over a period of days to months. The method preferably includes applying in sequence a series of two or more compositions that comprise an extract of Mimulus aurantiacus and a plant toxin, where the two or more compositions applied in sequence have increasing amount of the plant toxin.

### BRIEF DESCRIPTION OF SEVERAL VIEWS OF THE DRAWINGS

FIG. 1 is a flow chart outlining one method of making an extract of Mimulus aurianticus for use in treating psoriasis.

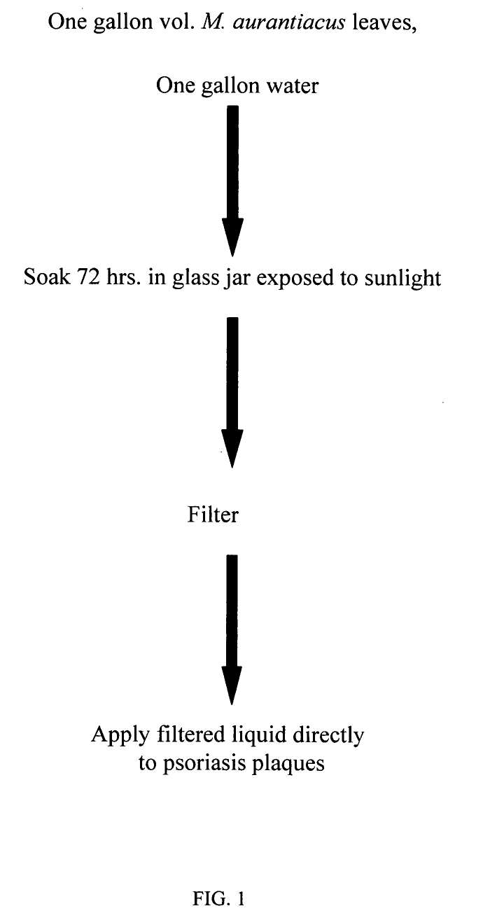

### DETAILED DESCRIPTION OF THE INVENTION

### Definitions

“Dermatitis” is inflammation of the skin. It includes contact dermatitis (including reactions to detergents, metals, plants (including, but not limited to, poison ivy (Toxicodendron rydbergii, Toxicodendron radicans) poison oak (Toxicodendron diversilobum, Toxicodendron toxicarium) and poison sumac (Toxicodendron vernix)), latex, etc.; seborrheic dermatitis, which can cause dandruff and cradle cap, perioral dermatitis, or atopic dermatitis.

“Tinea” is a fungal infection of the skin, hair, body, or nails. It includes athlete's foot, jock itch, and ringworm.

As used herein the term “insects” refers to members of the order Insecta, as well as other organisms belonging to the phylum Arthropoda, such as those belonging to the orders Arachnida (for example, spiders, scorpions, lice, ticks), Anoplura (sucking lice), and Siphonaptera (fleas).

I. Composition for Treating Skin Ailments

The present invention includes compositions comprising extracts of Mimulus aurantiacus that can be used to treat skin ailments. One skin ailment that can be treated using a composition of the present invention is psoriasis. Another skin ailment that can be treated using a composition of the present invention is eczema. Yet another skin ailment that can be treated with a composition of the present invention is dermatitis. Dermatitis can be any type of dermatitis, including but not limited to, seborrheic dermatitis, perioral dermatitis, atopic dermatitis, and contact dermatitis. Seborrheic dermatitis includes a scalp condition common to babies known as “cradle cap”. Contact dermatitis includes diaper rash, skin irritation due to chemicals, metals, or plant substances such as, but not limited to, poison ivy, poison oak, and poison sumac. Tinea, a fungus infection of the skin, hair, or nails that can cause athlete's foot, can also be treated with a composition of the present invention, as can acne, reactions to insect bites, and dandruff. These conditions share the characteristics of either persistent and abnormal skin cell build-up, or persistent skin inflammation (lasting hours to days) combined with any or all of the symptoms of rash, redness, itching, and pain.

Extraction Process

Extracts from the Mimulus aurantiacus plant can be extracts of the leaves, stems, flowers, or seeds. Preferably, an extract of the present invention uses the leaves of the Mimulus aurantiacus plant, optionally together with other parts of the plant. Whole plants or portions of plants can be used intact or can be sliced, minced, or mashed before making the extract.

Plant extracts may be most effective when made from plants that are harvested during the approximately six month period from December to June, a time when the leaf resin is extremely sticky, and the residual effect of the resin is at its peak. Plant material can optionally be harvested from plants during this period, and if desired, frozen for an extended period of time. The plants, or plant parts (for example, leaves, stems, or branches) can be frozen dry or in water.

An extract of Mimulus aurantiacus can be made by rinsing or soaking plants or portions of plants in a liquid, such as a liquid comprising water, at least one alcohol, acetone, or at least one oil. Other volatile chemicals can also be used for rinsing or soaking the leaves, however, liquids comprising water, alcohols, oils, (and combinations thereof) are preferred, because in most cases the volatile chemicals must be removed from the extract so that the final composition does not contains ingredients that may be irritating or harmful to the skin. Preferably, parts of the plant that include leaves are soaked in water or an alcohol, such as, for example, methanol, or a mixture of water and one or more alcohols. The liquid for soaking the leaves can also include other compounds or additives, such as but not limited to, surfactants, detergents, salts, antioxidants, reducing agents, chelators, acids, bases, buffering agents, oils, etc.

The ratio of plant parts to liquid is not limiting in making an extract of the present invention. Preferably, from approximately 0.5 grams to 40 grams of leaves is rinsed, or more preferably soaked, in one liter of water, one or more alcohols, or a mixture of water and one or more alcohols. In some preferred embodiments of the present invention, from approximately 2 grams to about 10 grams of M. aurantiacus leaves are soaked or rinsed with one liter of water, one or more alcohols, or a mixture of water and one or more alcohols. On a volume to volume basis, from approximately one cup to one half gallon of leaves is rinsed, or preferably soaked, in one gallon of water, one or more alcohols, or a mixture of water and one or more alcohols. More preferably, from approximately four cups to about one half gallon of M. aurantiacus leaves are soaked or rinsed in one gallon of water, one or more alcohols, or a mixture of water and one or more alcohols.

Rinsing can be performed by spraying a stream of liquid over the leaves, or by dunking the leaves in the liquid. Soaking is preferably but optionally performed in a closed container, and can be done from approximately two to 120 hours or longer. The container can optionally be turned, rotated, or shaken during the incubation. In some preferred embodiments of the present invention, soaking is performed in a closed container for approximately 72 hours.

Soaking can be done in the presence of light. The light can be of any wavelength, including the ultraviolet and infrared wavelengths. The light can be broad spectrum light, such as natural sunlight. In some preferred embodiments of the present invention, an extract of Mimulus aurantiacus is made by soaking leaves in water in a closed transparent container exposed to sunlight. The soaking leaves are exposed to sunlight during the daylight hours over three days (72 hours), after which time the liquid can be strained, filtered, or decanted. The liquid extract can be used directly as a salve, or can be further processed.

An extract of M. aurantiacus can also be made by heating leaves or other plant parts in a liquid. In these embodiments, from approximately 0.5 grams to 40 grams of leaves is added to one liter of liquid, preferably water or an aqueous solution. In some preferred embodiments of the present invention, from approximately 2 grams to about 10 grams of M. aurantiacus leaves heated in one liter of water or aqueous liquid. On a volume to volume basis, from approximately one cup to one half gallon of leaves is heated in one gallon of water or aqueous liquid. More preferably, from approximately four cups to about one half gallon of M. aurantiacus leaves heated in one gallon of water or an aqueous liquid. Heating is preferably performed at low temperature so that the mixture of leaves and liquid simmers over low heat. Preferably, the mixture is heated to less than or equal to 100 degrees Centigrade. The mixture of leaves and liquid can be heated for anywhere from twenty minutes to twenty-four hours or longer. Preferably, however, the mixture is heated from thirty minutes to twelve hours. After heating, the liquid extract can be strained, filtered, or decanted. The liquid extract can be used directly as a salve or spray, or can be further processed.

In one preferred embodiment, M. aurantiacus leaves are added to water or an aqueous solution, and the mixture is simmered over low heat in an open or closed vessel for two to twenty-four hours. This heating procedure can concentrate the extract. After cooling, the liquid is strained, filtered, or decanted and can be used directly as a salve or spray, or further processed.

An extract can also be made by soaking M. aurantiacus or portions thereof, in a liquid for a period of time, or by heating M. aurantiacus or portions thereof, in a liquid, and then soaking or heating an additional batch of M. aurantiacus in the first extract to get a “2× extract”. The 2× extract can be strained, filtered, or decanted and can be used directly as a salve or spray, or further processed.

Additional Processing Steps

Additional processing steps can also include concentrating the extract, such as by evaporation. In some preferred embodiments, the extract can be made by soaking plants or plant parts in a liquid (such as an aqueous solution or an alcohol-containing solution) as described above, and after the soaking period the liquid extract can be heated to reduce the volume. Lyophilization can also be used to concentrate the extract.

The extract can also be dried by evaporation (aided by heating) or lyophilization, down to a resin or powder, which can then be formulated into a composition for treating a skin ailment.

Subfractions of the extract can optionally be made, using chromatography, extraction with solvents, or other separation and purification methods as they are known in the field of organic chemistry. The extract can also be a purified or partially purified fraction of a crude extract of Mimulus aurantiacus. For example, various fractionation procedures, including but not limited to phase separation, differential precipitation, or chromatography can be used to partially or essentially purify a fraction of the extract that comprises the desirable activity. Optionally, the one or more chemical entities of the partially or essentially purified material of the active fraction can be identified using chemical techniques such as, but not limited to, NMR or mass spectrometry. The partially or essentially purified material of the active fraction can optionally be quantified using chemical and biophysical methods.

Such fractions or preparations can be tested for their efficacy in treating skin ailments using tests known in the art, such as, for example, the Dermatology Life Quality Index (DLQI) the Psoriasis Symptom Assessment (PSA) Scale, the Visual Analog Scale (VAS) and the National Psoriasis Foundation (NPF) itch measure.

At any point during the making of an extract other compounds can be added. Such compounds include, but are not limited to, buffering agents, acids, bases, salts, thickeners, antioxidants, reducing agents, chelators, detergents, surfactants, oils, waxes, paraffin, or additional plant extracts.

Formulations

The present invention encompasses compositions that include extracts of M. aurianticus The composition can be formulated as a lotion, cream, salve, gel, lotion, foam, spray, soap, or shampoo. In formulating a composition, thickeners, gelling agents, foaming agents, surfactants, oils, waxes, paraffin, emollients (moisturizers), emulsifiers, humectants, smoothing agents, sunblocking or sunscreening agents, detergents, polymers, alcohols (including benzyl alcohol), and the like can be added. The composition can also comprise other additives such as, but not limited to, acids, bases, buffering agents, preservatives, metals (such as, but not limited to, zinc oxide and titanium oxide), chelators, antioxidants, benzyl benzoate, vitamins, coloring agents, perfumes, etc. A composition of the present invention can also have other ingredients that can have therapeutic value, such as, but not limited to, extracts of other plants.

Additional plant extracts can be any plant extracts, in crude or highly purified form. For example, extracts of aloe vera (Aloe barbadensis), marigold (calendula officinalis), chamomile (Anthemis nobilis), laurel (Taurus nobilis), oregon grape, cucumber (Curcumis sativus) cornflower (Centaurea cyanus), green tea (Camellia sinensis), ginseng (Panax ginseng), Wrightia tinctoria, Eucalyptus globulus, Primula veris, Sambucus nigra, Helianthus annuus, Chamomila recutita, as well as coconut oil, palm oil, jojoba oil, compounds extracted from olive leaves (oleuropein) and Oregon grapeseeds (berberine) etc. are among the extracts that can be useful in compositions of the present invention. (See for example, U.S. Pat. No. 4,235,889; U.S. Pat. No. 5,858,372; U.S. Pat. No. 6,225,342; and U.S. Pat. No. 6,440,465; all herein incorporated by reference in their entireties.)

Compositions of the present invention can contain from to 0.5% to 100% of M. aurantiacus extracts. For example, in some preferred embodiments of the present invention, a spray or salve to treat psoriasis can comprises 100% of M. aurantiacus extract. In other embodiments of the present invention, a cream, salve, shampoo, gel or foam for treating cradle cap can comprise 50% or less of M. aurantiacus extract. The strength and overall compositions of formulations can be tested for efficacy using standard tests known in the pharmaceutical and personal care product industries. For example, studies can be performed using multiple subjects having the same skin ailment, where one group of subjects receives applications of the M. aurantiacus formulation, and the other group of subjects receives applications of the formulation that lack the of M. aurantiacus extract. Preferably, these studies are double-blind studies, in which neither the practitioner nor the patients know which formulations contain the M. aurantiacus extract. Where practical, studies can also be done in which a single subject suffering from a skin ailment receives treatments with a M. aurantiacus formulation on one affected area of the skin, and treatments with a placebo formulation on another affected area of the skin. After the treatment period, which can last from hour to months, the condition of the areas is compared. These tests can be performed on animals or on people. Where the test subjects are people, subjective as well as objective measures of improvement can be taken into account. Tests for the efficacy of formulations in treating skin ailments are known in the art, and the choice of tests will depend on the skin condition being treated. For example, the Dermatology Life Quality Index (DLQI) the Psoriasis Symptom Assessment (PSA) Scale, the Visual Analog Scale (VAS) and the National Psoriasis Foundation (NPF) itch measure can be used.

II. Methods of Treating a Skin Ailment Using a Composition of the Present Invention that Comprises an Extract of M. aurantiacus

The present invention includes methods of using a composition of the present invention that includes an extract of M. aurantiacus to treat a skin condition. The method includes administering the composition that comprises an extract of M. aurantiacus to the affected area of skin of a subject. The subject can be a human or an animal.

The means of administration will largely depend on the formulation. For example, salves, creams, gels and oils can be smoothed on the affected area of the skin. Foams and sprays can be applied by pumps or aerosol (pressure-release) spray, and optionally smoothed on, or in some cases, rubbed into, the affected area. Soaps and shampoos can be used for washing skin or hair.

The frequency of administration can depend on the strength of the formulation, the severity of the condition, and other factors, such as the age of the subject. Those skilled in the art of dermatology can readily determine dosages and administration regimes. Preferably, a composition of the present invention is administered at a frequency of from three times per day to twice a week.

Other Applications

A composition of the present invention that comprises an extract of Mimulus aurantiacus can also be used as a sunscreen. Many flavonoids, including some of those isolated from the surface of Mimulus aurantiacus leaves, absorb in the UV range, and thus, if applied to the skin, can prevent harmful wavelengths of sunlight from reaching the surface of the skin. The present invention also includes composition that comprises an extract of Mimulus aurantiacus that can be used as a sunscreen. Such compositions can be applied as creams, lotions, gels, or sprays.

III. Composition for Repelling Insects Comprising an Extract of Mimulus aurantiacus

The present invention also includes compositions comprising extracts of Mimulus aurantiacus that can be used to repel insects or herbivores. As used herein, “insects” includes not only members of the order Insecta, but also members of the order Arachnida (for example, spiders, scorpions, lice, ticks), Anoplura (sucking lice), and Siphonaptera (fleas). Herbivores include any plant eating animals, including but not limited to, nematodes, snails, rabbits, woodchucks (also called marmots or groundhogs), and deer.

Extraction Process

Extracts from the Mimulus aurantiacus plant can be extracts of the leaves, stems, flowers, or seeds. Preferably, an extract of the present invention uses the leaves of the Mimulus aurantiacus plant, optionally together with other parts of the plant. Plants, or portions thereof, can be used intact or can be sliced, minced, or mashed before making the extract.

Plant extracts may be most effective when made from plants that are harvested during the approximately six month period from December to June, a time when the leaf resin is extremely sticky, and the residual effect of the resin is at its peak. Plant material can optionally be harvested from plants during this period, and if desired, frozen for an extended period of time. The plants, or plant parts (for example, leaves, stems, or branches) can be frozen dry or in water.

An extract of Mimulus aurantiacus can be made by rinsing or soaking plants or portions of plants in a liquid, such as a liquid comprising water, at least one alcohol, acetone, or at least one oil. Other volatile chemicals can also be used for rinsing or soaking the leaves, however, liquids comprising water, alcohols, oils, (and combinations thereof) are preferred, because in most cases the volatile chemicals must be removed from the extract so that the final composition does not contains ingredients that may be harmful to plants, animals, or humans. Preferably, parts of the plant that include leaves are soaked in water or an alcohol (such as, for example, methanol), or a mixture of water and one or more alcohols. The liquid for soaking the leaves can also include other compounds or additives, such as but not limited to, surfactants, salts, antioxidants, chelating agents, acids, bases, oils, etc.

The ratio of plant parts to liquid is not limiting in making an extract of the present invention. Preferably, from approximately 0.5 grams to 40 grams of leaves is rinsed, or preferably soaked, in one liter of water, one or more alcohols, or a mixture of water and one or more alcohols. More preferably, from approximately 2 grams to about 10 grams of M. aurantiacus leaves are soaked or rinsed with one liter of water, one or more alcohols, or a mixture of water and one or more alcohols. On a volume to volume basis, from approximately one cup to one half gallon of leaves is rinsed, or preferably soaked, in one gallon of water, one or more alcohols, or a mixture of water and one or more alcohols. More preferably, from approximately four cups to about one half gallon of M. aurantiacus leaves are soaked or rinsed in one gallon of water, one or more alcohols, or a mixture of water and one or more alcohols.

Rinsing can be performed by spraying a stream of liquid over the leaves, or by dunking the leaves in the liquid. Soaking is preferably but optionally performed in a closed container, and can be done from approximately two to 120 hours or longer. The container can optionally be turned, rotated, or shaken during the incubation. In some preferred embodiments of the present invention, soaking is performed in a closed container for 72 hours.

Soaking can be done in the presence of light. The light can be of any wavelength, including the ultraviolet and infrared wavelengths. The light can be broad spectrum light, such as natural sunlight. In some preferred embodiments of the present invention, an extract of Mimulus aurantiacus is made by soaking leaves in water a closed transparent container exposed to sunlight. The soaking leaves are exposed to sunlight during the daylight hours over three days (72 hours), after which time the liquid can be strained, filtered, or decanted and can be used directly as an insect repellant, or further processed.

An extract of M. aurantiacus can also be made by heating leaves or other plant parts in a liquid. In these embodiments, from approximately 0.5 grams to 40 grams of leaves is added to one liter of liquid, preferably water or an aqueous solution. In some preferred embodiments of the present invention, from approximately 2 grams to about 10 grams of M. aurantiacus leaves heated in one liter of water or aqueous liquid. On a volume to volume basis, from approximately one cup to one half gallon of leaves is heated in one gallon of water or aqueous liquid. More preferably, from approximately four cups to about one half gallon of M. aurantiacus leaves heated in one gallon of water or an aqueous liquid. Preferably the mixture of leaves and liquid is simmered over low heat. Preferably, the mixture is heated to less than or equal to 100 degrees Centigrade. The mixture of leaves and liquid can be heated for anywhere from twenty minutes to twenty-four hours or longer. Preferably, however, the mixture is heated from thirty minutes to twelve hours. After heating, the liquid extract can be strained, filtered, or decanted. The liquid extract can be used directly as a salve or spray, or can be further processed.

In another preferred embodiments, M. aurantiacus leaves are added to water or an aqueous solution, and the mixture is simmered over low heat in an open or closed vessel for two to twenty-four hours. After cooling, the liquid can be strained, filtered, or decanted and can be used directly as an insect repellant, or further processed.

It is also possible to make extracts by allowing plants or plant parts to at least partially ferment. During a field test, a volume of approximately 25 gallons of compressed Mimulus aurantiacus plants were put in a 55 gallon drum. The drum was then filled with water and the material was left outside to naturally ferment over the course of three weeks. Five nearby drums were filled with water but contained no plant material. At the end of the three week period, all five water-filled drums contained larvae, including mosquito larvae. The drum that contained the Mimulus plants had no larvae. The fermentation process may intensify anti-insect properties of Mimulus plants. The liquid obtained after fermentation can be optionally strained and used as an insect repellant.

Additional Processing Steps

Additional processing steps can also include concentrating the extract, such as by evaporation. In some preferred embodiments, the extract can be made by soaking plants or plant parts in a liquid (such as an aqueous solution or an alcohol-containing solution) as described above, and after the soaking period the filtered extract can be heated to reduce the volume. Lyophilization can also be used to concentrate the extract.

The extract can also be dried by evaporation (aided by heating) or lyophilization, down to a resin or powder. The resin or powder can be provided as is or formulated with additional compounds to be reconstituted as a liquid by the user. The reconstituted liquid can be used as a salve or spray for repelling insects.

Subfractions of the extract can optionally be made, using chromatography, extraction with solvents, or other separation and purification methods as they are known in the field of organic chemistry. Such fractions can be tested for their efficacy in repelling insects using tests such tests that tabulate the number of insect bites on the treated and untreated arms of subjects (See, for example, Fradin and Day (2002) New Engl J Med 347: 13-18), or the amount of insect damage to the leaves of treated and untreated plants.

At any point during the making of an extract other compounds can be added. Such compounds include, but are not limited to, buffering agents, acids, bases, salts, antioxidants, reducing agents, chelators, surfactants, detergents, oils, or additional plant extracts.

Formulations

The present invention encompasses compositions that include extracts of M. aurianticus The composition can be formulated as a lotion, cream, salve, gel, lotion, foam, spray, soap, or shampoo.

In some aspects of the present invention, a composition of the present invention is used to repel insects or herbivores from plants. In these aspects, formulations can be liquids that can be sprayed on plants and in the vicinity of plants.

Formulations for use in repelling insects from plants can also be resins or dry powders or granules that are easy to store and can be mixed with water for spraying. In this case, an extract can be dried down by evaporation or lyophilization. The evaporation or lyophilization can optionally be done in the presence of other chemical that can improve the consistency or dissolvability of the dried-down product. Dry or highly concentrated compounds (such as salts, or surfactants or detergents in concentrated or solid form) can also optionally be mixed with the dried-down extract to increase ease of use or its efficacy as an insect or herbivore repellant.

In other aspects of the present invention, a composition of the present invention is used to repel insects from people or animals. Such formulations can be lotions, creams, gels, foams, sprays, shampoos, or soaps. In formulating a composition, thickeners, gelling agents, foaming agents, surfactants, oils, waxes, paraffin, emollients (moisturizers), emulsifiers, humectants, smoothing agents, sunblocking or sunscreening agents, detergents, polymers, alcohols (including benzyl alcohol), and the like can be added. The composition can also comprise other additives such as, but not limited to, acids, bases, buffering agents, preservatives, benzyl benzoate, metals (such as, but not limited to, zinc oxide and titanium oxide), chelators, antioxidants, vitamins, coloring agents, perfumes, etc. A composition of the present invention can also have other ingredients that can have therapeutic value, such as, but not limited to, extracts of other plants.

Additional plant extracts can be any plant extracts, in crude or highly purified form. As nonlimiting examples, extracts of aloe vera (Aloe barbadensis), chamomile (Anthemis nobilis), laurel (laurus nobilis), cornflower (Centaurea cyanus), green tea (Camellia sinensis), ginseng (Panax ginseng), Wrightia tinctoria, Eucalyptus globulus, Primula veris, Sambucus nigra, Helianthus annuus, Chamomila recutita, as well as coconut oil, palm oil, jojoba oil, etc. are among the extracts that can be useful in compositions of the present invention that can be applied to the skin, such as the skin of humans or animals. Extracts of other plants, such as, but not limited to citronella, cedar, peppermint, lemongrass, geranium, soybean, and eucalyptus can also be added to a composition of the present invention for repelling insects from animals and humans to enhance its efficacy.

Among the additional plant extracts that can be useful in a formulation for use in repelling insects from plants are extracts (such as extracts of the leaves) of poison ivy (Toxicodendron rydbergii, Toxicodendron radicans) poison oak (Toxicodendron diversilobum, Toxicodendron toxicarium) and poison sumac (Toxicodendron vernix). For example, leaves of poison oak can be extracted using a solution that includes at least one detergent. Once treated with detergent, the leaves and their extracts are no longer toxic to humans. The detergent extract can be added to an insect-repelling formulation that comprises Mimulus aurantiacus extract to increase its insect-repelling properties. Extracts of other plants, such as, but not limited to, citronella, cedar, peppermint, lemongrass, geranium, soybean, eucalyptus and chrysanthemums (including purified pyrethrins) can also be added to a composition of the present invention for repelling insects from plants to enhance its efficacy.

In yet other aspects of the present invention, an extract of M. aurianticus can make up essentially the entire composition used to repel insects from plants. An extract of Mimulus aurantiacus, optionally concentrated, can be directly applied or sprayed on plants or animals.

III. Methods of Using an Extract of Mimulus aurantiacus to Repel Insects or Herbivores

Methods for Repelling Insects and Herbivores from Plants

The present invention includes methods of using a composition of the present invention to repel insects or herbivores from a plant. In most applications, a formulation for plant use will be in the form of a liquid, or made into a liquid by dissolving granules, and the liquid is sprayed or “watered” onto a plant. The spraying or watering can be performed as a single application or multiple times. In some preferred embodiments, the application is repeated at least once, after an interval of from eight to twenty-four hours.

The composition can be applied to plants to repel insects or herbivores, such as, but not limited to, snails, deer, and rabbits.

In another preferred embodiment of the present invention, the extract comprises ground up Mimulus aurantiacus plants (or portions of plants, for example, the leaves), and the “mulch” is spread around the base of plants to deter crawling insects, nematodes, snails, and the like.

Other Applications

A composition of the present invention that comprises an extract of Mimulus aurantiacus can also be used to protect plants form exposure to excessive UV light. Many flavonoids, including some of those isolated from the surface of Mimulus aurantiacus leaves, absorb in the UV range, and thus, if applied to the surfaces of leaves, can prevent harmful wavelengths of sunlight from penetrating the leaves. The present invention also includes compositions that comprise an extract of Mimulus aurantiacus that can be used to protect plants from excessive UV light, such as when a plant is transferred to a high light environment to which it is not adapted. Such compositions can be applied as sprays or watering solutions.

Methods for Repelling Insects and Herbivores from Animals

The present invention includes methods of using a composition of the present invention to repel insects from a human or animal. A formulation for repelling insects can be in the form of a lotion, shampoo, soap, spray, cream, salve, foam, or gel. In some preferred aspects of the present invention, the formulation is a lotion, spray, cream, salve, or gel that is applied to the skin of a human to repel insects such as mosquitos, ticks, deer flies, and black flies. In other preferred aspects of the present invention, the formulation is a soap or shampoo that can be used in washing to repel or eradicate lice or other insects. In yet other preferred embodiments, the formulations are used on pets or other animals to repel or eradicate insects. For example, Liquids can be sprayed on pets or livestock to deter insects. Foams or gels can be rubbed into the fur of a dog or cat for repelling or eradicating ticks or fleas. Shampoos can also be used on pets for the same purpose. The schedule of applications of insect-repelling formulations to human or animal can be determined empirically. The schedule of applications of sprays, lotions or creams will also depend on such factors as the activity level of the subject.

IV. Compositions Comprising an Extract of Mimulus aurantiacus and a Plant Toxin

The present invention also includes compositions and methods for increasing the tolerance of an individual to a toxin, such as a plant toxin, such as, for example, a toxin of poison ivy (Toxicodendron (or Rhus) rydbergii, Toxicodendron (or Rhus) radicans) poison oak (Toxicodendron (or Rhus) diversilobum, Toxicodendron (or Rhus) toxicarium) or poison sumac (Toxicodendron (or Rhus) vernix). In these aspects, the inventors contemplate that tolerance of an individual to a plant can be increased by graduated exposure to the toxin. The invention includes compositions that can be applied to the skin of an individual that include an extract of Mimulus aurantiacus and at least one plant toxin. The Mimulus aurantiacus extract provides a soothing effect to the skin that ameliorates the irritation of the toxin while the individual is being exposed to the toxin by topical application of the lotion, cream, gel, salve, foam, or spray.

The compositions include: at least one plant toxin and an extract of Mimulus aurantiacus. The compositions preferably also include at least one emollient that can aid in the spreadability of the composition and reduce skin irritation and dryness.

An extract of Mimulus aurantiacus can be made as described in Section I and the Examples of the present application. The extract can comprise from 0.5% to 95% of a Mimulus aurantiacus. The extract of can also be a partially or essentially purified fraction of a crude extract of Mimulus aurantiacus.

The plant toxin can be any plant toxin, but preferably is a plant toxin from the leaves, stems, branches, or roots of poison ivy (Toxicodendron (or Rhus) rydbergii, Toxicodendron (or Rhus) radicans) poison oak (Toxicodendron (or Rhus) diversilobum, Toxicodendron (or Rhus) toxicarium) or poison sumac (Toxicodendron (or Rhus) vernix). Urushiol, the toxin produced by these plants, is soluble in alcohol, acetone, and detergent solutions. It can be extracted from the leaves, stems, or roots of poison oak, poison ivy, or poison sumac plants.

Methods of extracting urushiol from plants are known in the art and can be found, for example, in Corbett and Billets (1975) J. Pharm Sci. 64 (10); 1715-1718 and Craig et al. (1978) J. Pharm Sci. 67 (4); 483-485. A lotion, cream, gel, foam, or spray of the present invention for inducing tolerance to urushiol can comprise one or more catechol derivatives. One or more of the urushiols can be chemically synthesized or a derivative of a naturally occurring urushiol.

Preferably, the urushiol used in a composition of the present invention is substantially purified and quantified, so that a known quantity of urushiol is added to a composition of the present invention. For example, a lotion, cream, gel, or spray that comprises an extract of Mimulus aurantiacus and urushiol can comprise from about 0.001 picogram to about 1 milligram of urushiol per ounce (thirty milliliters), and preferably comprises from about 0.005 picogram to about 100 micrograms of urushiol per ounce (thirty milliliters).

In preferred embodiments of the present invention, lotions, creams, gels, foams, or sprays for inducing tolerance to urushiol are provided having different concentrations of urushiol. For example, the concentration of urushiol in a “weak” formulation can be 0.005 picogram per ounce (thirty milliliters), the concentration of urushiol in a “moderate” formulation can be 0.01 nanogram per ounce (thirty milliliters), and the concentration of urushiol in a “strong” formulation can be 1 nanogram per ounce (thirty milliliters). These examples are not intended to be limiting in any way. The optimal amount and combinations of urushiol in formulations of the present invention can be optimized based on the amount of formulation to be applied to the skin, the testing of formulations, and the sensitivity of an individual subject to urushiols. Where practical, studies can also be done in which a single subject suffering from a skin ailment receives treatments with a M. aurantiacus formulation on one affected area of the skin, and treatments with a placebo formulation on another affected area of the skin. After the treatment period, which can last from days to weeks, the condition of the areas is compared. These tests can be performed on animals or on people. Where the test subjects are people, subjective as well as objective measures of improvement can be taken into account.

Formulations

The present invention encompasses compositions that include extracts of M. aurianticus and at least one urushiol. Compositions of the present invention can contain from to 0.5% to 99% of M. aurantiacus extracts. The M. aurantiacus extract can alleviate irritation due to the urushiols while still allowing exposure of the individual to the urushiols for the induction of tolerance. The optimal amount of M. aurantiacus extract to be used can be determined empirically.

The composition can be formulated as a lotion, cream, salve, gel, foam, or spray. In formulating a composition, thickeners, gelling agents, foaming agents, surfactants, oils, waxes, paraffin, emollients (moisturizers), emulsifiers, humectants, smoothing agents, sunblocking or sunscreening agents, detergents, polymers, alcohols (including benzyl alcohol), and the like can be added. The composition can also comprise other additives such as, but not limited to, acids, bases, buffering agents, preservatives, metals (such as, but not limited to, zinc oxide and titanium oxide), chelators, antioxidants, benzyl benzoate, vitamins, coloring agents, perfumes, etc. A composition of the present invention can also have other ingredients that can have therapeutic value, such as, but not limited to, extracts of other plants.

Additional plant extracts can be any plant extracts, in crude or highly purified form. For example, extracts of aloe vera (Aloe barbadensis), marigold (calendula officinalis), chamomile (Anthemis nobilis), laurel (Taurus nobilis), oregon grape, cucumber (Curcumis sativus) cornflower (Centaurea cyanus), green tea (Camellia sinensis), ginseng (Panax ginseng), Wrightia tinctoria, Eucalyptus globulus, Primula veris, Sambucus nigra, Helianthus annuus, Chamomila recutita, as well as coconut oil, palm oil, jojoba oil, compounds extracted from olive leaves (oleuropein) and Oregon grapeseeds (berberine) etc. are among the extracts that can be useful in compositions of the present invention.

V. Methods of Using a Composition Comprising an Extract of Mimulus aurantiacus and a Plant Toxin to Increase the Tolerance of an Individual to a Plant Toxin

The present invention further includes methods of increasing tolerance of individual to a toxin, such as a plant toxin. The method includes applying a composition comprising an extract of Mimulus aurantiacus and a plant toxin to the skin of an individual and monitoring the individual for sensitivity to the plant toxin. Preferably, the plant toxin is a urushiol, such as but not limited to a urushiol of poison ivy (Toxicodendron (or Rhus) rydbergii, Toxicodendron (or Rhus) radicans) poison oak (Toxicodendron (or Rhus) diversilobum, Toxicodendron (or Rhus) toxicarium) or poison sumac (Toxicodendron (or Rhus) vernix). Preferably, the composition is applied over a period of days to months. Preferably, two or more compositions comprising an extract of Mimulus auranliacus and a plant toxin are applied to the skin of an individual in sequence, where the compositions comprise different concentrations of the plant toxin, and the composition with the lowest concentration of plant toxin is applied first, and compositions comprising higher concentrations of plant toxin are applied in order of increasing toxin concentration. In this way, the individual's tolerance to a plant toxin is increased, while the Mimulus aurantiacus extract in the formulations ameliorates the irritation caused by the toxin.

For example, an individual may receive a single application of a cream, lotion, gel, foam, or spray of the present invention having a low concentration of a plant toxin to the skin of the arm, and one to two weeks later receive a single application of a cream, lotion, gel, foam, or spray of the present invention having a somewhat higher concentration of the plant toxin to the same or a different area of the skin. Successively higher concentration formulations can be applied over weeks or months to produce tolerance to the toxin in the individual. Two, three, or more formulations can be employed, where the formulations have different concentrations of toxin and the patient receives applications of formulations of increasing concentration over time.

Treatment regimens can be determined empirically. For example, application of a formulation of the present invention can be one or more times a day, or less often (for example, weekly). A composition of a given strength (toxin concentration) can be applied once or more than once. Various dosage increments can be employed.

The amount of formulation applied to the skin, as well as the amount of skin area treated, can also be determined empirically.

Treatment regimens can be tested on animals, for example, mice and guinea pigs, to test their effectiveness, before testing on humans (see, for example, U.S. Pat. No. 5,643,571, herein incorporated by reference and Stampf et al. (1986) Journal of Investigative Dermatology 86(5): 535-538, herein incorporated by reference).

### EXAMPLES

### Example 1

### Making an Extract of Mimulus aurantiacus

Leaves were stripped from Mimulus aurantiacus plants to fill a one gallon container. The leaves were placed in a glass jar, and one gallon of water was poured over them. The lid was screwed onto the container, and the jar was left outside, exposed to direct sunlight, for three days (72 hours) in Escondido, Calif. Daytime temperatures ranged from 56 to 92 degrees Fahrenheit. Nighttime temperatures ranged from 48 to 65 degrees Fahrenheit.

After incubating the leaves in water in the presence of natural sunlight, the jar was opened. The liquid was a rich brown color. The mixture was strained, and the strained liquid was kept in a closed jar in the refrigerator.

### EXAMPLE 2

### Use of a Composition Comprising an Extract of M. aurantiacus to Treat Psoriasis

The strained liquid from Example 1 is used directly to test its effects on psoriasis. One arm of a single subject that exhibited psoriasis in the elbow region is slathered liberally with the composition. The application is performed three times per day for a period of six weeks. The other arm of the subject does not receive any treatment. The degree of redness, scaling, and itching experienced by each arm is recorded daily.

### EXAMPLE 3

### Compositions for Repelling Insects Comprising M. aurantiacus Extracts.

Leaves are stripped from Mimulus aurantiacus plants to fill a one gallon container. The leaves are placed in a two-gallon capacity glass jar, and one gallon of water is poured over them. The lid is screwed onto the container, and the jar is left outside, exposed to direct sunlight, for three days (72 hours) in Escondido, Calif. Daytime temperatures range from 56 to 92 degrees Fahrenheit. Nighttime temperatures range from 48 to 65 degrees Fahrenheit.

After incubating the leaves in water in the presence of natural sunlight, the jar is opened. The mixture is strained, and the strained liquid is returned to the glass jar. Additional fresh leaves are added (amount that fills a one gallon container) to the jar, and the jar is returned to direct sunlight for an additional three day (72 hour) incubation. After the second incubation, the “2× Extract” is strained, and kept in a closed jar in the refrigerator.

### EXAMPLE 4

### Use of a Composition Comprising an Extract of M. aurantiacus to Repel Insects

The composition of Example 3 is poured into a pump spray bottle. The composition is sprayed liberally over six basil plants in pots. An additional six basil plants in pots are sprayed with water. The pots containing the basil plants are set outside together. Each day for a week the plants are sprayed at the same time.

At the end of the week, insect damage on each plant is inspected.

Plants sprayed with extract have reduced damage from insects, evidenced by more leaves, and more intact leaves.

All publications, including patent documents and scientific articles, referred to in this application, including any bibliography, are incorporated by reference in their entirety for all purposes to the same extent as if each individual publication were individually incorporated by reference.

All headings are for the convenience of the reader and should not be used to limit the meaning of the text that follows the heading, unless so specified.

1. A composition for the topical treatment of a skin ailment comprising an extract of the leaves, stems, roots, flowers, seeds, or a combination thereof of Mimulus aurantiacus.

2. The composition of claim 1, wherein said skin ailment is selected from the group consisting of psoriasis, eczema, tinea, athlete's foot, acne, reaction to insect bites, diaper rash, cradle cap, dandruff, seborrheic dermatitis, perioral dermatitis, atopic, dermatitis, and contact dermatitis.

3. The composition of claim 1, wherein said extract is made by rinsing or soaking the leaves, stems, roots, flowers, seeds, or combination thereof of at least one Mimulus aurantiacus plant in a liquid comprising water, at least one oil, acetone, or at least one alcohol.

4. The composition of claim 3, wherein said extract is made by soaking the leaves of Mimulus aurantiacus plant in water.

5-11. (canceled)

12. The composition of claim 1, wherein said composition comprises from about 0.5% to about 100% of said extract.

13. The composition of claim 12, wherein said extract is formulated as a lotion, shampoo, soap, spray, cream, salve, foam, or gel.

14. The composition of claim 13, further comprising at least one emollient, oil, wax, surfactant, detergent, moisturizer, antioxidant, or additional plant extract.

15. A method of treating a skin ailment, comprising administering the composition of claim 1 to the skin of a subject.

16. The method of claim 15, wherein said skin ailment is selected from the group consisting of psoriasis, eczema, tinea, athlete's foot, acne, reaction to insect bites, diaper rash, cradle cap, dandruff, seborrheic dermatitis, perioral dermatitis, atopic, dermatitis, and contact dermatitis.

17. (canceled)

18. A composition for repelling insects or herbivores comprising an extract of the leaves, stems, roots, flowers, seeds, or combination thereof, of Mimulus aurantiacus.

19. The composition of claim 18, wherein said extract is made by rinsing or soaking the leaves, stems, roots, flowers, seeds, or combination thereof of at least one Mimulus aurantiacus plant in a liquid comprising water, at least one oil, acetone, or at least one alcohol.

20. The composition of claim 19, wherein said extract is made by soaking the leaves of Mimulus aurantiacus plant in water.

21-26. (canceled)

27. The composition of claim 18, wherein said composition comprises from about 0.5% to about 100% of said extract.

28. The composition of claim 18, wherein said composition repels insects.

29. The composition of claim 28, wherein said composition is formulated as a formulated as a lotion, spray, shampoo, soap, cream, salve, foam, or gel.

30-34. (canceled)

35. A method of repelling or eradicating insects comprising applying the composition of claim 18 to an animal or human subject.

36. A method of repelling insects or herbivores comprising applying the composition of claim 18 to a plant.

37. A composition for increasing the tolerance of an individual to a plant toxin comprising at least one plant toxin and an extract of the leaves, stems, roots, flowers, seeds, or a combination thereof of Mimulus aurantiacus.

38. The composition of claim 37, formulated as a lotion, cream, gel, foam, or spray.

39. The composition of claim 38, wherein said at least one plant toxin is urushiol.

40-49. (canceled)
